(19) United States
(12) Patent Application Publication
(10) Pub. No.: US 2021/0071249 A1
(43) Pub. Date: Mar. 11, 2021

(54) DIAGNOSTIC METHOD AND THERAPY

(71) Applicant: Oxford University Innovation Limited, Dxford (GB)

(72) Inventors: Sarosh R. IRANI, Oxford (GB); Patrick WATERS, Oxford (GB); James VARLEY, Oxford (GB); Melanie RAMBERGER, Oxford (GB)

(21) Appl. No.: 17/051,930

(22) PCT Filed: May 7, 2019
(86) PCT No.: PCT/GB2019/051257

§ 371 Date: Oct. 30, 2020

(30) Foreign Application Priority Data
May 4, 2018 (GB) 1807410.4

(51) Int. Cl. C12Q 1/6881; G01N 33/564; C07K 7/08; A61K 38/00; A61P 37/06

(52) U.S. Cl. CPC C12Q 1/6881; G01N 33/564; C07K 7/08; G01N 2800/24; A61P 37/06; G01N 2800/52; C12Q 2600/106; A61K 38/00

(57) ABSTRACT

The invention relates to a method of identifying patients who are positive for CASPR2 autoantibodies which are predicted to respond to immunotherapy, the method comprising i) obtaining a sample from a subject having autoantibodies against CASPR2; and ii) screening for the presence of the allele HLA DRB1*11:01. The invention also relates to one or more novel isolated peptides, wherein the peptide comprises the sequence of any of Seq ID no: 1 to Seq ID no: 57 or a sequence having at least 80%, 85%, 90%, 95% or more sequence identity with one of Seq ID no 1 to 7, preferably with one of Seq ID no 40 to 57.

The invention relates to an improved method for diagnosing autoimmune diseases in mammals, and in particular to an improved method for stratifying subjects to ensure the most appropriate therapy is given. The invention also provides therapeutic peptides for use in treating autoimmune diseases.

Voltage-gated potassium channel (VGKC) antibodies are associated with four main clinical syndromes: neuromyotonia (NMT), Morvan's syndrome (MoS), seizures and limbic encephalitis (LE). Other syndromes are increasingly recognised including movement disorders such as ataxia and myoclonic syndromes, pain syndromes and forms of epilepsy (Becker et al., 2012; Gadoth et al., 2017; Podewils et al., 2017). NMT describes peripheral nerve hyperexcitability syndromes causing muscle cramps and stiffness, and sometimes pain. MoS describes NMT plus autonomic features, for instance excessive sweating, constipation, cardiac irregularities, and central nervous system features, particularly confusion, hallucinations and insomnia. LE associated with anti-VGKC antibodies includes the central nervous system (CNS)-restricted features of amnesia, personality or psychiatric disorders, and seizures (epilepsy). The seizures can occur in isolation. These conditions (particularly MoS) can be associated with thymic or other tumours (lung carcinoma, lymphoma, gynaecological malignancies) but anti-VGKC antibody associated LE is mainly non-paraneoplastic. All four syndromes have a subacute onset and may be immunotherapy-responsive. Most patients so far are adults, but some children with these antibodies and LE or epilepsy have been identified. As already demonstrated in vivo with NMT, much evidence supports a pathogenic role of LE and MoS immunoglobulin G (IgG). Firstly, patients often experience a prompt clinical recovery following plasma exchange. Secondly, the antibody titres in an individual patient correlate well with alterations in clinical state. Thirdly, the patient IgG binds the hippocampus, the anatomical region to which many of their CNS clinical features can be localised.

In recent years the discovery of autoantibodies against leucine-rich, glioma-inactivated 1 (LGI1), contactin-associated protein-like 2 (CASPR2) (Irani et al., 2010; Lai et al., 2010) and, more recently, intracellular epitopes of voltage gated potassium channels (VGKCs) (Lang et al., 2017), have redefined the immunology of the VGKC-complex. Patient stratification by these antigenic targets has shown that the ‘double-negative’VGKC-complex antibodies, those without LGI1- or CASPR2-reactivities, are observed across all ages, in healthy controls and in a variety of syndromes, many of which are not immune-mediated. In contrast, patients with LGI1- or CASPR2-antibodies often have clinically-indistinguishable forms of limbic encephalitis (LE) and neuromyotonia with associated dysautonomia, sleep disturbances, pain and seizures. While these features occur at different rates in LGI1- versus CASPR2-antibody cohorts, only faciobrachial dystonic seizures (FBDS) robustly predict LGI1-reactivity. Furthermore, these two autoantibodies are both often of the IgG4-subclass, testing for these directly can be more sensitive than testing for VGKC-complex antibodies (Becker et al., 2012; Irani et al., 2013), and frequently coexist in patients with the ultra-rare Morvan's syndrome. The striking overlaps of these rare neurological features and autoantibodies, and the frequent co-expression of their antigenic targets within mammalian CNS-membrane complexes (Binks et al., 2017; Irani et al., 2010), suggest they are involved in autoimmunisation. The nature of the available complexes, antigen presentation mechanisms and the available T cell repertoires are likely to determine which antigen dominates the ensuing T-B cell response. If so, human leucocyte antigen (HLA) variants, intimately related to antigen presentation, may play critical roles in distinguishing the aetiology of these syndromes. HLA variants may also allow patients to be stratified into different groups and offered the most appropriate therapy.

Currently, when diagnosed as positive for autoantibodies against CASPR2, patients are frequently administered immunotherapy. Yet, it is recognised that a number of patients with CASPR2 antibodies do not have immunotherapy-responsive, or immune-mediated, neurological syndromes (Bien et al., 2017). In our clinical practice, there have been several such examples. Administration of such immunotherapies is, therefore, not always effective and it is also very costly and has a number of potential side-effects. It would therefore be advantageous to be able to identify which patients with autoantibodies against CASPR2 are most likely to respond to immunotherapy, and thus target immunotherapy more appropriately.

The present invention provides a combination of a serological test and a genetic test to improve the diagnosis of autoimmune neurological disorders where CASPR2 autoantibodies are likely to be causative of disease, and where patients are most likely respond to immunotherapies. More specifically, the method of the invention combines serological analysis to determine the presence of CASPR2 autoantibodies in a subject, and genetic analysis of a subject to determine the presence of a particular HLA allele. In particular, the invention provides a method of identifying patients who are positive for CASPR2 autoantibodies which are predicted to respond to immunotherapy, the method comprises:
i) obtaining a sample from a subject having autoantibodies against CASPR2; and
ii) screening for the presence of the allele HLA DRB1*11:01.

Preferably the method further comprises the step of concluding that if the HLA DRB1*11:01 allele is present then immunotherapy should be administered. In the presence of the HLA DRB1*11:01 allele, CASPR2 autoantibodies are assumed to be causative of disease and taking action with immunotherapy, to neutralise or eliminate them or the cells which produce them, is expected to improve the subjects condition.

The method of the invention allows subjects with CASPR2 autoantibodies to be stratified into those which are predicted to respond to immunotherapy—that is, those which have the HLA DRB1*11:01 allele, and those that are predicted not to respond to immunotherapy—that is, those which do not have the HLA DRB1*11:01 allele. For subjects which have CASPR2 autoantibodies but do not have the HLA DRB1*11:01 allele further tests may be undertaken to determine the most appropriate diagnosis and therapy.

According to another aspect, the invention provides a method of diagnosing in a mammal an autoimmune neurological disorder comprising:
i) obtaining a sample provided by a subject;
ii) detecting the presence or absence of CASPR2 autoantibodies in the sample;
iii) detecting the presence of the HLA DRB1*11:01 allele in the patient; and optionally
iv) concluding that if CASPR2 autoantibodies are present and the HLA DRB1*11:01 allele is present that the subject has an autoimmune neurological disorder, likely to be mediated by CASPR2-antibodies and likely to respond to immunotherapy.

The autoimmune neurological disorder may be selected from the group consisting of limbic encephalitis, Morvan's syndrome, seizures and neuromyotonia, and other increasingly recognised associations of CASPR2-antibodies, including movement disorders such as ataxia and myoclonic syndromes, pain syndromes and forms of epilepsy. Where the neurological disorder is limbic encephalitis, the dominant feature of the neurological disorder may comprise seizures, for example epilepsy, or amnesia or psychiatric disorder alone.

Preferably the method of the invention is performed in combination with an assessment of clinical symptoms. The combination of the method of the invention and an analysis of clinical symptoms may be used to determine the specific neurological disorder an individual has.

In addition to screening for the HLA DRB1*11:01 allele and/or CASPR2 autoantibodies, the method of the invention may also comprise screening for one or more of the following:
the titre of CASPR2 autoantibodies;
the IgG subclass of CASPR2 autoantibodies;
the level of CASPR2 autoantibodies in solution, which may be determined by a fluorescent immunoprecipitation assay (FIPA);
the level of autoantibodies to the C and/or N termini of CASPR2.

By screening for one or more of the above an even greater likelihood of diagnosing an immunotherapy-responsive disease can be achieved.

If the titre of CASPR2 autoantibodies is high, for example is greater than 1:2000 using cell based assays, then it may concluded that the autoantibodies are disease causing and the subject should be treated accordingly, typically with immunotherapy. The titre level of CASPR2 autoantibodies alone may be used as a diagnostic tool, that is, without determining if the HLA DRB1*11:01 allele is present. Alternatively both the titre of CASPR2 autoantibodies and the presence of the HLA DRB1*11:01 allele may be used, for example in an algorithmic approach to diagnostic testing.

If the titre of CASPR2 autoantibodies is low, that is between about 1:100 and 1:2000 using cell based assays, then it may concluded that further studies should be undertaken to determine if the autoantibodies are disease causing. Other tests which may be considered include determining if the HLA DRB1*11:01 allele is present, determining the subclass of the IgG CASPR2 autoantibodies and/or determining the level of CASPR2 autoantibodies in solution (FIPA).

The CASPR2 autoantibodies may be detected by any immunological assay technique, of which many are well known in the art. Examples of suitable techniques include ELISA, radioimmunoassay, fluoroimmunoassay, a competition assay, an inhibition assay, a sandwich assay, spectrometry, western blot, protein microarray, surface enhanced Raman spectroscopy, isoelectric focusing and the like. In general terms, such assays use an antigen, which may be immobilised on a solid support. A sample to be tested is brought into contact with the antigen and if autoantibodies specific to the antigen are present in a sample they will immunologically react with the antigen to form autoantibody antigen complexes, which may then be detected or quantitatively measured. Alternatively, the antigen can be expressed on the surface or within a cell which is permeabilised. Detection of autoantibody-antigen complexes may be carried out using a secondary anti-human immunoglobulin antibody, typically anti-human IgG or anti-human IgM, which recognises general features common to all human IgGs or IgMs respectively. The secondary antibody is usually conjugated to an enzyme such as, for example, horseradish peroxidise (HRP), so that detection of an antigen/autoantibody/secondary antibody complex may be achieved by addition of an enzyme substrate and subsequent colorimetric, chemiluminescent or fluorescent detection of the enzymatic reaction products, or it may be conjugated to a fluorescent signal. Preferably the method uses a secondary antibody which is a tagged or labelled anti-human IgG antibody. Preferably the anti-human IgG antibody is labelled with a reporter molecule. The reporter molecule may by a heavy metal, a fluorescent or luminescent molecule, a radioactive tag or an enzymatic tag. An enzymatic tag may be HRP.

Preferably the intensity of the signal from the anti-human IgG antibody is indicative of the relative amount of the CASPR2 autoantibody in the bodily fluid when compared to a positive or negative control.

If the subclass of the CASPR2 autoantibodies is IgG4, this is indicative that the autoantibodies are causative of disease.

If anti-human CASPR2 antibody is observed in solution in a subject's serum then this is indicative that the autoantibodies are causative of disease. The level of CASPR2 autoantibodies in solution, may be determined by a FIPA. To determine the level of anti-human CASPR2 antibody in solution, CASPR2 covalently linked to EGFP is expressed in HEK cells and then solubilised with detergent. This extract is then incubated with patient sera and precipitated either with Protein-G (which binds IgG 1-4), or anti-human IgG secondary to form a pellet. The EGFP precipitated is then read as a measure of the anti-human CASPR2 antibody level in the serum.

In addition to screening for CASPR2 autoantibodies, the method of the invention may also include the step of screening for LGI1 autoantibodies. These tests are frequently, and in many centres routinely, requested alongside one another in a clinical setting as many of the clinical syndromes are indistinguishable, and several patients have both coexisting CASPR2 and LGI1-antibodies.

Immunotherapy may include the administration of one or more of therapeutic antibodies, chemotherapy, intravenous immunoglobulins, steroids, cytokines, plasma exchange therapy, adoptive cell therapy, CAR T-cell therapy and any other suitable immunotherapy.

Side effects of immunotherapy may include flu like symptoms, complete with fever, chills, and fatigue. Others side effects could include swelling, weight gain from extra fluids, heart palpitations, nausea or vomiting, diarrhoea, muscle or joint aches, fatigue, low or high blood pressure, breathing difficulties and dizziness. Subjects may also experience skin reactions at the site of injection, such as pain, swelling, soreness, redness, itchiness and rash.

The sample used in the method of the invention may be a bodily fluid. The bodily fluid may comprise plasma, serum, whole blood, urine, sweat, tears, saliva, lymph, faeces, cerebrospinal fluid, or nipple aspirate. Preferably the bodily fluid is serum or plasma.

The method of the invention may include the step of taking the sample from the subject. Alternatively, the method of the invention may not include the step of taking the sample, but instead the sample may be provided after it has been taken from the subject.

The method of the invention may be carried out in vitro.

The subject may be a human.

In an another aspect the invention provides a method for predicting whether or not an individual will respond to immunotherapy, wherein the method comprises determining whether a subject has CASPR2 autoantibodies and whether a subject has the HLA DRB1*11:01 allele, and if both are present it is predicted that the subject will respond to immunotherapy.

In an alternative embodiment of any aspect of the invention, as an alternative to, or in addition to, considering whether a subject has the HLA DRB1*11:01 allele the relative presence of autoantibodies to the N and/or C termini of CASPR2 may be used to predict a subject will respond to immunotherapy.

In a further aspect, the invention provides a method of treating an autoimmune neurological disorder in an individual, the method comprising:
i) diagnosing an autoimmune neurological disorder according to the method above; and
ii) administering to the individual an agent useful in the treatment of the autoimmune neurological disorder.

The agent may be immunotherapeutic agent, alternatively or additional the agent may be a peptide as described herein.

In a still further aspect the invention provides a system comprising:
a) a measuring module for determining the presence of CASPR2 autoantibodies in a biological sample from a subject;
b) a measuring module for determining the presence of the HLA DRB1*11:01 allele in a biological sample from a subject.
c) a storage module configured to store data output from the measuring module or modules, and optionally reference data;
d) a computation module configured to compute the value of the data output from the measuring module or modules, and optionally the reference data; and
e) an output module configured to display a diagnosis for the subject based on the results obtained by the computation module.

According to a further aspect, the invention provides an assay kit for diagnosing, in a mammal, an autoimmune neurological disorder selected from the group comprising limbic encephalitis, Morvan's syndrome, neuromyotonia, and other conditions according to the method of the invention, wherein the kit comprises at least one epitope of CASPR2 that is recognised by CASPR2 autoantibodies and primers for use in detecting the HLA DRB1*11:01 allele, and instructions to use the kit.

Preferably, the kit also comprises means for contacting the at least one epitope of CASPR2 with a bodily fluid sample from a mammal.

According to a further aspect, the invention provides one or more novel isolated peptide. The peptide may comprise the sequence of any of Seq ID no: 1 to Seq ID no: 57, the peptide may comprise the sequence of any of Seq ID no: 40 to 57, the peptide may comprise the sequence of any of Seq ID no: 50 to 57. The peptide may bind to an MHC molecule encoded by an HLA allele observed in subjects which have LG11 autoantibodies or CASPR2 autoantibodies. The peptide may be based on the CASPR2 or the LG11 protein. The peptide may be 30 amino acids long or less, may be 25 amino acids or less, may be 20 amino acids or less. The peptide may be between 5 and 30 amino acids, between 10 and 30 amino acids, between 5 and 25 amino acids, between 10 and 25 amino acids, between 15 and 25 amino acids, between 5 and 20 amino acids, between 10 and 20 amino acids or between 15 and 20 amino acids. The peptide may comprise the sequence of any of Seq ID no: 1 to 57, the peptide may comprise the sequence of any of Seq ID no: 40 to 57, with 1, 2, 3, 4, 5, 6, 7, 8, 9, 10 or more amino acids on either the C-terminus or the N-terminus, or on both termini. The peptides of the invention may comprise a sequence having at least 80%, 85%, 90%, 95% or more sequence identity with one of Seq ID no 1 to 57, preferably with one of Seq ID no 40 to 57.

The percent identity of two amino acid sequences is generally determined by aligning the sequences for optimal comparison purposes (e.g. gaps can be introduced in the first sequence for best alignment with the second sequence) and comparing the amino acid residues at corresponding positions. The “best alignment” is an alignment of two sequences that results in the highest percent identity. The percent identity is determined by comparing the number of identical amino acid residues within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990), modified as in Karlin and Altschul (1993). The NBLAST and XBLAST programs of Altschul et al. (1990) have incorporated such an algorithm. To obtain gapped alignments for comparison purposes, Gapped BLAST can be utilized as described in Altschul et al. (1997). Alternatively, PSI-Blast can be used to perform an iterated search that detects distant relationships between molecules. When utilizing BLAST, Gapped BLAST, and PSI-Blast programs, the default parameters of the respective programs (e.g. XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

The peptide may be acetylated, acylated, alkylated, glycosylated, and the like. The peptide may include non-natural amino acids.

The peptide may be part of a fusion protein.

The peptide may include one or more conservative amino acid substitutions as compared to the sequences given for Seq ID no: 1 to 57.

The peptide may be isolated from a natural system or may be synthetically or recombinantly produced.

The peptide may be straight or cyclic. The peptide may include a protease resistant backbone. The peptide may include modifications at the C- and/or N-terminus. The peptide may be labelled, such as with a radioactive label or a fluorescent label.

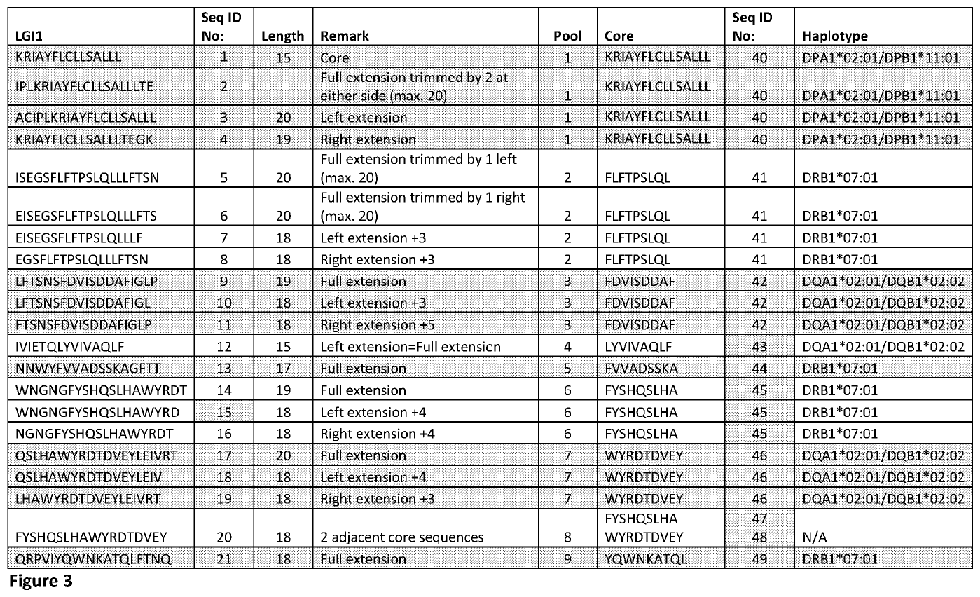

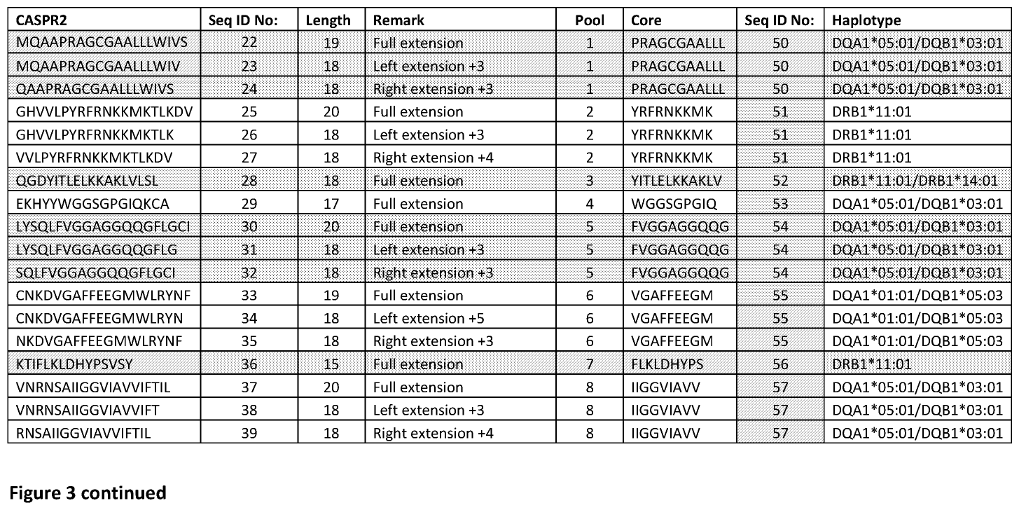

The peptide of the invention may be for use in the treatment of subject with an autoimmune neurological disorder. The autoimmune neurological disorder may be limbic encephalitis, Morvan's syndrome, seizures or neuromyotonia, or others including movement disorders such as ataxia and myoclonic syndromes, pain syndromes and forms of epilepsy. The subject may have LG11 and/or CASPR2 autoantibodies. The subject may have an HLA allele which expresses an MHC molecule to which it is predicted the peptide will bind—as summarised in the table in FIG. 3.

The invention may further comprise a pharmaceutical composition comprising a peptide of the invention and a pharmaceutically acceptable carrier, diluent or excipient. In an embodiment, at least 5% of the pharmaceutical composition is a carrier, diluent or excipient.

A “pharmaceutically acceptable” carrier or excipient, as used herein, means approved by a regulatory agency, such the FDA or MHRA, or as listed in the U.S. Pharmacopoeia or other generally recognized pharmacopoeia for use in mammals, and more particularly in humans.

The carrier may, for example, be water or an aqueous fluid such as saline. However, the skilled person will be well aware of carriers, diluents or excipients that are pharmaceutically acceptable.

The pharmaceutical composition may also comprise one or more of a buffering agent, a viscosity-increasing agent, a solvent, a stabiliser and a preservative.

In a further aspect, the invention provides a method of treating subject with an autoimmune neurological condition comprising administering to the subject a therapeutically effective amount of a peptide or a pharmaceutical composition according to the invention.

In a further aspect, the invention provides a method of treating a subject with an autoimmune neurological condition caused by CASPR2 autoantibodies, the method comprising administering to the subject an effective amount of a peptide according to the invention comprising the sequence of any one of Seq ID nos: 19 to 39 and 50 to 57.

In a further aspect, the invention provides a method of treating a subject with an autoimmune neurological condition caused by LG11 autoantibodies, the method comprising administering to the subject a therapeutically effective amount of a peptide according to the invention comprising the sequence of any one of Seq ID nos: 1 to 18 and 40 to 49.

The route of administration of the peptide or pharmaceutical composition may be injection or infusion by parenteral, subcutaneous, intravenous, intramuscular, intraperitoneal, intraarterial, intralesional, intraarticular, topical, oral, rectal, nasal, inhalation or any other suitable route.

The dosage of the peptide used will depend on the peptide, the target and the treatment. The determination of the dosage and route of administration is well within the skill of an ordinary physician.

The skilled man will appreciate that any of the preferable features discussed above can be applied to any of the aspects or embodiments of the invention. Many equivalent modifications and variations will be apparent to those skilled in the art. Various changes to the described embodiments may be made without departing from the scope of the invention.

The references described herein are incorporated by reference.

Preferred embodiments of the present invention will now be described, merely by way of example, with reference to the following figures and examples.

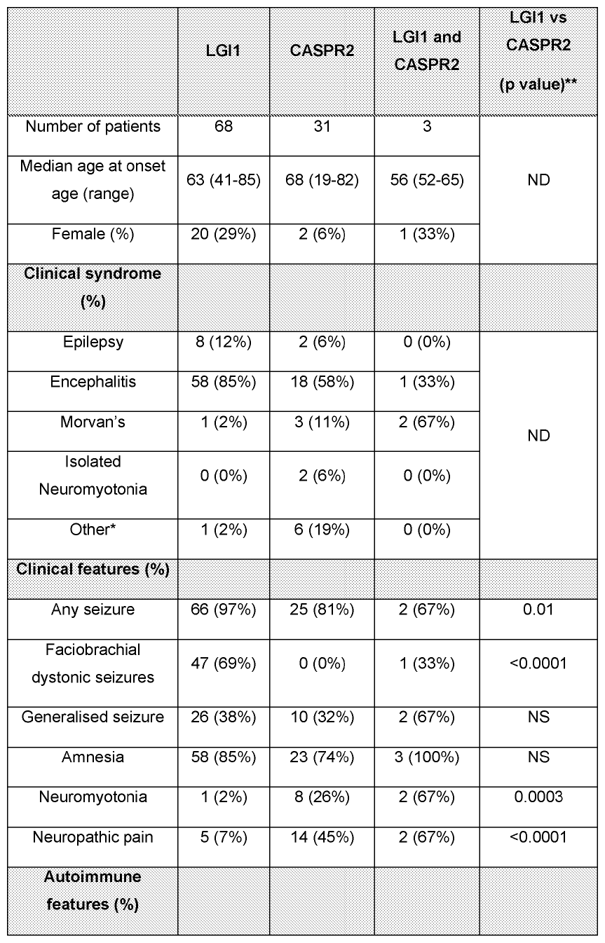

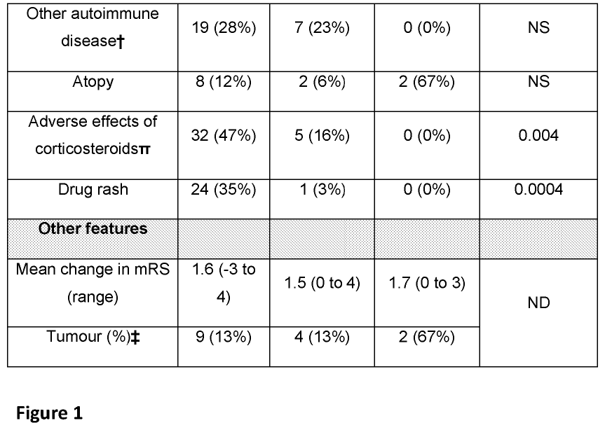

FIG. 1—is a table illustrating the clinical features of patients with antibodies to VGKC complex proteins: LGI1, CASPR2 and both LGI1 and CASPR2. Live cell based assays were used for LGI1- and CASPR2-antibody determination (Irani et al., 2010). *Other diagnoses included movement disorders (n=4, CASPR2, generalised chorea, hemifacial spasm, cervical dystonia and cerebellar ataxia), axonal neuropathy (n=1, CASPR2), psychosis (n=1, CASPR2) and stroke (n=1 with LGI1-antibodies). *Statistical comparisons with Fisher's exact test throughout. † Autoimmune diseases in LGI1-antibody patients: [n=19: diabetes (n=1), heparin-induced thrombocytopaenia (n=1), hyper- and hypothyroidism and Hashimoto's thyroiditis (n=8), multiple sclerosis (n=1), myasthenia gravis (n=1), neuromyelitis optica (n=1), optic neuritis (n=1), pernicious anaemia (n=1), psoriasis (n=6), Raynaud's disease (n=1), and ulcerative colitis (n=1)] and CASPR2-antibody patients [n=7: congenital adrenal hyperplasia, hypothyroidism, pernicious anaemia, pemphigus, polymyalgia rheumatic, psoriasis and Raynaud's disease (all n=1)]. Corticosteroid-related complications, sometimes multiple, in LGI1-antibody patients [n=32: marked weight gain (n=12), behavioural disturbance (n=5) and diabetes (n=5), or worsened diabetes (n=1), insomnia (n=4), fracture (n=3), myopathy or muscle weakness (n=3), skin thinning/easy bruising (n=3), mania/hypomania (n=2), poor wound healing or abscess (n=2), ophthalmic infections (n=2; keratitis and ophthalmic shingles), perforated abdominal viscus (n=2), and one each of: avascular necrosis of the hip (AVN), cerebral venous sinus thrombosis, high INR and steroid-induced psychosis] and in CASPR2-antibody patients [n=5: marked weight gain (n=1), rash (n=2), striae/thin skin/bruising (n=2), and hallucinations (n=1)]. ‡ Tumours in LGI1-antibody patients (n=9) were: basal cell carcinoma (n=3), other skin—type not known (n=2), bladder (n=1), breast (n=1), prostate (n=1), dysplastic colonic polyp (n=1) and in 4 CASPR2-antibody patients were: pancreatic (n=1), prostate (n=2), thymic cyst (n=1). NS=not significant; ND=not done; mRS=modified Rankin scale (Thompson et al., 2018).

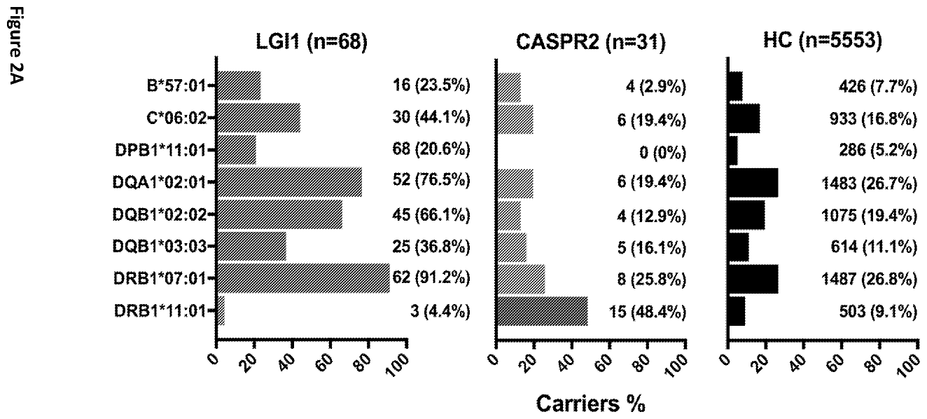

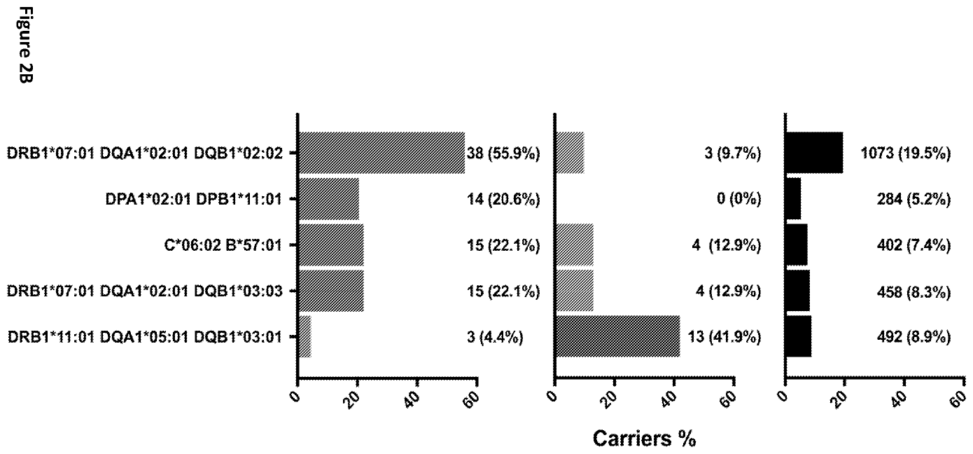

FIG. 2—demonstrates HLA allele and haplotype associations in patients with LGI1- and CASPR2-antibodies. The bar chart depicts allele (A) and haplotype (B) associations and their frequency in patients with antibodies to LGI1 (n=68, significant associations are observed with all alleles depicted except DRB1*11:01) and CASPR2 (n=31, significant associations are observed with DRB1*11:01), together with the frequency of these alleles or haplotypes in 5553 healthy controls (HC).

FIG. 3—is a table detailing peptides derived from LG11 and CASPR2 sequence and HLA binding partners. The peptides from LG11 and CASPR2 that are predicted to bind the HLA variants derived from in silico haplotype analyses are presented. The positions of the peptide clusters within the full-length molecule (15 mer starting position), the extended core amino sequence, the highest affinity of the peptides in the cluster (nM), and the predicted binging to LG11 and CASPR2-cohort haplotypes are given.

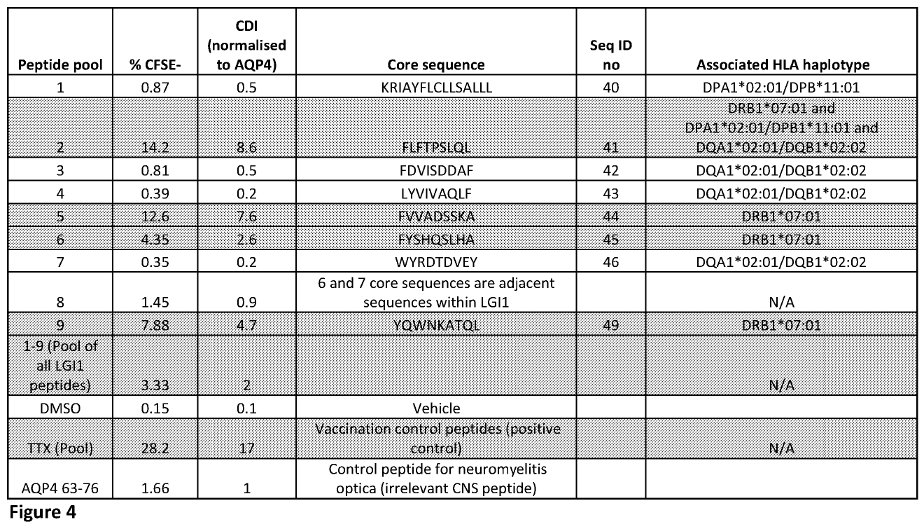

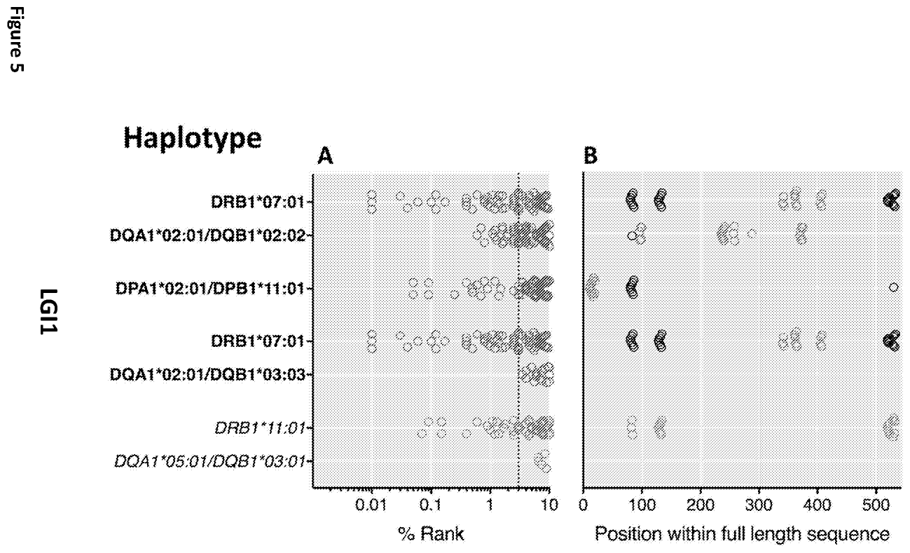

FIG. 4—details LGI1 peptides that have been tested in vitro. CD4+ proliferation was defined as cell division index (CDI) normalised to an irrelevant CNS peptide (AQP4) ≥2. In one patient heterozygous for DRB1*07:01, the CDI of multiple LGI1 peptides predicted to bind DRB1*07:01 were above the cut-off (≥2, highlighted rows). TTX=tetanus toxin (a positive control, as most people have anti-tetanus CD4 T cells). AQP4=aquaporin 4: a CNS antigen in patients with a different antibody-mediated illness.

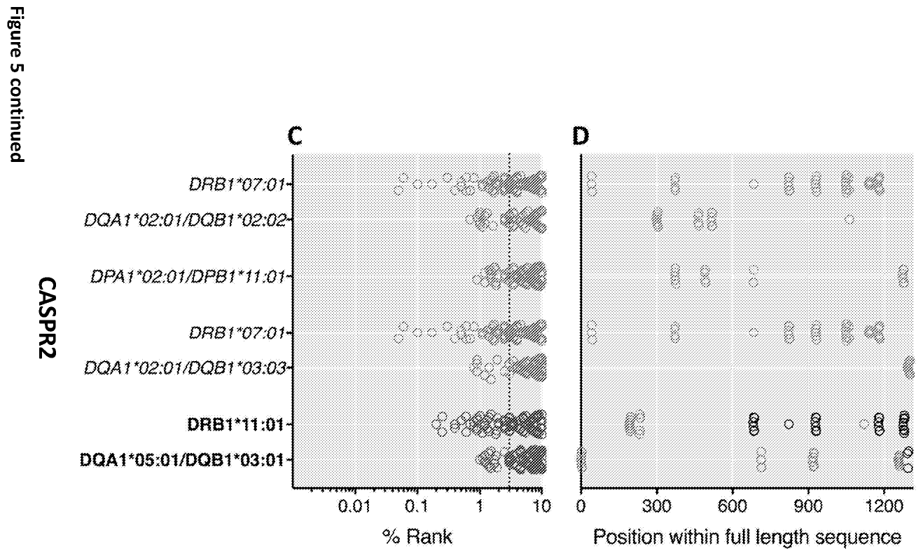

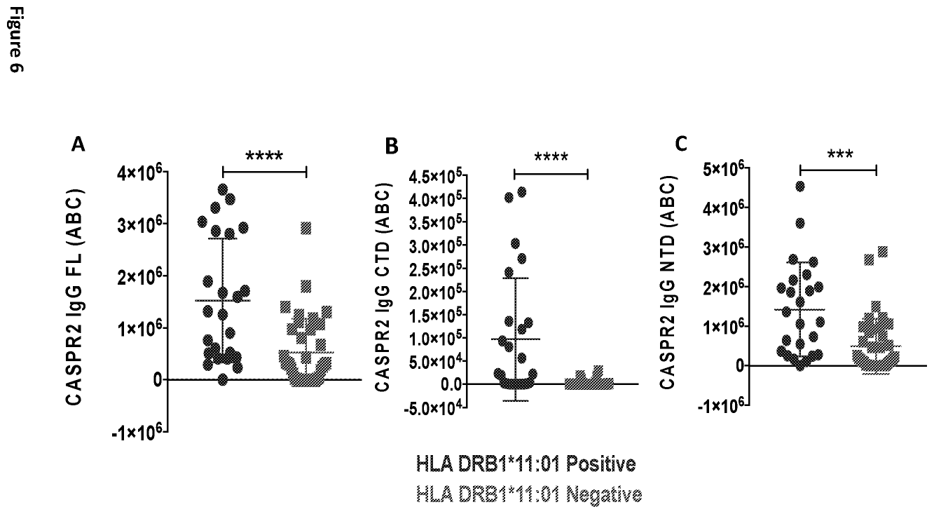

FIG. 5—illustrates peptides derived from full-length LGI1 and CASPR2 predicted to bind MHC-dimers encoded by overrepresented HLA haplotypes. Rankings and the position of peptides derived from full-length sequences of LGI1 (A, B) and CASPR2 (C, D) are illustrated. The haplotypes correspond to FIG. 2B and when in bold they relate to those observed in patients with antibodies to the corresponding protein. Red circles denote the LGI1-antibody cohort and blue the CASPR2-antibody cohort. Grey circles and italicised haplotypes relate to peptides from the other antigenic protein (i.e. CASPR2 in A and B; and LGI1 in C and D). Rank describes the predicted peptide affinities (IC50, nM) by comparison to 200,000 random peptides of the same length. Dotted lines represent the 3% cut-off for peptide rank. Within B and D, circles represent the highly-ranked peptides across the full-length sequences of LGI1 or CASPR2: black circles represent peptides with some predicted promiscuity across LGI1 and CASPR2-antibody HLA-variants, whereas purple circles highlight peptides which are not predicted to cross-react.

FIG. 6—illustrates that the overall CASPR2 antibody titre (FIG. 6A; FL=to full length CASPR2) is higher in patients with HLA-DRB1*11:01. Such patients also demonstrate a significantly higher titre of antibodies against the C- and N-termini of CASPR2 (CTD and NTD, respectively, FIG. 6B-C).

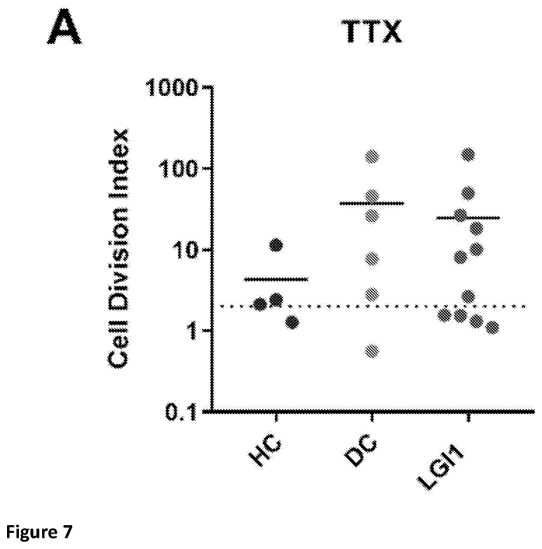

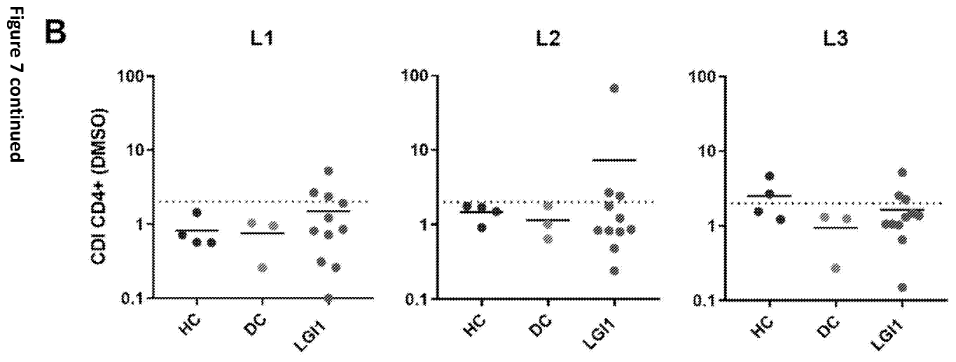

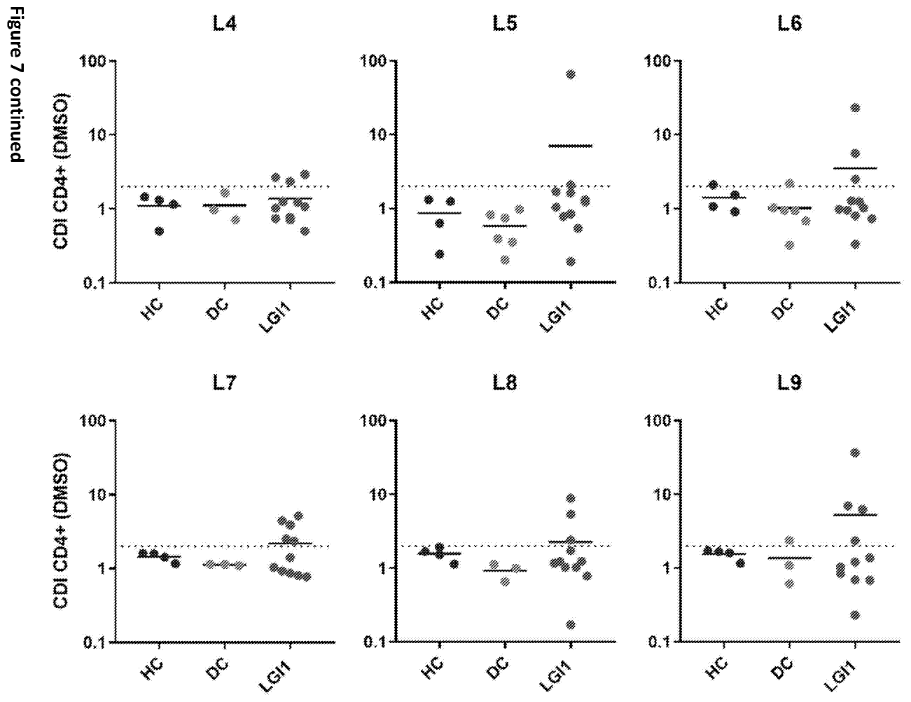

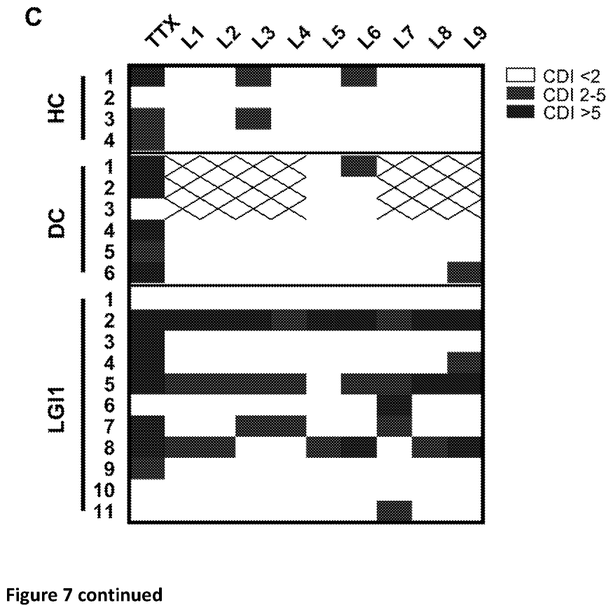

FIG. 7—summarizes the CD4⁺ T cell responses to positive control tetanus toxin TTX peptides (FIG. 7A) and individual LGI1 peptides and pools (FIG. 7B) as described in FIG. 3. The cut-off CDI (≥2) is indicated by a dotted line. FIG. 7C shows the antigen-specific CD4⁺ T cell responses of individual patients/controls. No response (CDI<2, white), intermediate proliferation (CDI 2-5, light blue) and strong proliferation (CDI>5, dark blue) are indicated by coloured cells, missing values are indicated by X. CDI: cell division index. DC: disease controls. HC: healthy controls. LGI1: patients with LGI1 antibodies. TTX: tetanus toxin.

### MATERIALS AND METHODS

### Patients

One hundred and eleven Caucasian patients were identified from previous studies (n=51) (Irani et al., 2011; 2013; Lang et al., 2017), referrals to the Oxford Autoimmune Neurology Group (n=49) or from the Autoimmune Encephalopathy clinic, University of California San Francisco (n=11). All had serum antibodies against LGI1 only (n=68), CASPR2 only (n=31), both LGI1 and CASPR2 (n=3) or intracellular aspects of VGKCs (n=9), as determined by previously described antigen-specific cell-based assays (Irani et al., 2010; Lang et al., 2017). Clinical phenotypes, including information relating to past medical history and adverse drug reactions (ADR)s (FIG. 1), were evaluated via direct patient and relative interviews and case-note reviews. All patients provided written informed consent (REC16/YH/0013 or the IRB 10-04905 approvals).

### Clinical Samples

Serum samples were obtained from patients and stored at −20° C.

### Detecting CASPR2 Autoantibodies

CASPR2 autoantibodies are detected using a live cell based assay. HEK293 cells are engineered to express CASPR2 on their surface, and then exposed to patient sera. The binding of any antibodies in the sera to CASPR2 expressed on the HEK293 cell surface is determined using immunohistochemistry. More specifically, HEK293 cells were cultured in Dulbecco's modified Eagle's medium (DMEM) supplemented with 10% foetal calf serum (FCS, TCS Cellworks Ltd, Buckingham, UK) and 100 units/ml each of penicillin G and streptomycin (Invitrogen, CA, USA) at 37° C. in a 5% CO₂ atmosphere. Cells were grown on 13 mm glass coverslips in 6-well cell culture plates for microscopy. Using polyethylenimine (PEI), cells were transiently transfected with CASPR2-EGFP cDNA. The expression of EGFP was visualised using an Axion 200 inverted Zeiss fluorescence microscope.

24 hours post-transfection immunofluorescent staining of HEK cells was performed. Coverslips were incubated in 24-well culture plates, which contained patient sera (1:20-8000) diluted in DMEM-N-(2-hydroxyethyl)piperazine-N′-(2-ethanesulphonic acid) (HEPES) with 1% bovine serum albumin (BSA), at room temperature (RT) for 1 hour. Cells were subsequently washed 3 times in DMEM-HEPES buffer and fixed with 3% formaldehyde in phosphate buffered saline (PBS) at RT for 15 minutes. Cells were washed as above and labelled for 45 minutes at RT with anti-human IgG Alexa Fluor 568-conjugated secondary antibody (Invitrogen-Molecular probes, Paisley, UK) at 1:750 in 1% BSA-DMEM-HEPES buffer. Cells were subsequently washed 3 times in PBS and mounted on slides in fluorescent mounting medium (DakoCytomation, Cambridge, UK) with DAPI (4′,6′-diamidino-2-phenlindoledichloride, 1:1000). They were visualised using a fluorescence microscope with a MacProbe v4.3 digital imaging system.

In order to determine the titre of CASPR2 autoantibodies in a plasma sample, the sample was diluted 1:X with saline or cell media or another suitable diluent. The titre is then given as the highest dilution (X) at which CASPR2 autoantibodies can be detected—the more CASPR2 autoantibodies in a sample the more it can be diluted and antibodies can still be detected.

### Detecting the Presence of the HLA-DRB1*11:01 Allele

The presence of the HLA-DRB1*11:01 allele was determined by PCR, and in particular by using sequence specific primers PCR (SSP-PCR) as per (Bunce et al., 1995.

### Identifying Possible Therapeutic Peptides Based on HLA Binding Predictions

The NetMHCIIpan 3.1 server model based on artificial neural-networks (Andreatta et al., 2015) evaluated HLA-haplotype binding affinities for 15 amino acid-long consecutive overlapping peptides from full-length LGI1 and CASPR2 sequences (UniProt accession numbers 095970 and Q9UHC6, respectively). Predicted peptide affinities (nM) were compared to 200,000 random peptides of the same length to generate rank values: this measure is less susceptible to the intrinsic capacity of some HLA-alleles to generate high-affinity predictions, and rank values (%)<3 were considered strong binders. As expected, consecutive 15-mer peptides with high rank values often shared a core sequence.

### Testing the Efficacy of Identified Peptides

A proliferation assay was used to test whether peptides predicted in silico to bind to the identified HLAs actually do. In the assay, peripheral blood mononuclear cells (PBMCs) were isolated from whole blood using Ficoll gradient reagent and immediately used for subsequent experiments. At a density of 2×10⁷ cells per ml isolated PBMCs were stained with 0.4 μM CFSE following the manufacturer's instructions with minor modifications. Briefly, PBMCs were stained with CFSE diluted in PBS for ten minutes at 37° C. The reaction was stopped by adding RPMI-1640 growth medium containing 10% FCS. The cell suspension was placed on ice for ten minutes, and after one further washing step with RPMI-1640 containing 10% FCS, followed by one washing step with RPMI-1640 without FCS, the cells were cultivated in X-Vivo 15 growth medium (Lonza, Basel, Switzerland). For the expansion of antigen-specific T cells, PBMCs were exposed to LGI1 peptide pools at a final concentration of 10 μg/ml (Peptides&Elephants, Potsdam, Germany) based on their predicted high binding affinity for patient associated HLA haplotypes (FIG. 4). As controls, a tetanus toxin (TTX) pool and an irrelevant CNS peptide (AQP463-76) were used. As vehicle control, dimethyl sulfoxide (DMSO) was used. Cells were seeded at a density of 2×10⁵ cells per 200 μl in round bottom 96-well tissue culture test plates, each six wells per condition. After eight days cells were re-stimulated with either peptide pools (10 μg/ml per peptide pool) or vehicle control and 100 μl of the supernatant were replaced with fresh medium containing 20 U/ml IL-2. Three days later, after 11 days in culture, PBMC were analyzed by flow cytometry. To determine the proliferation of T cells, PBMC were stained with CD3 and CD4 antibodies and analyzed on an LSR II flow cytometer. For analysis of a positive T cell proliferation response, the cell division index (CDI) was calculated as follows:

(CD3+CD4+CFSE−cells stimulated with LGI1 peptides (%))/(CD3+CD4+CFSE−cells stimulated with an irrelevant CNS peptide(AQP4)(%)).

A CDI ≥2 was considered as significant proliferation.

### Results for Diagnostic Method and the Importance of the HLA-DRB1*11:01 Allele

### Clinical Differences Between Patients Stratified by VGKC-Complex Autoantibody Targets

FIG. 1 summarises the clinical features of 111 patients, sub-grouped by their autoantibody specificities. In agreement with previous studies, onset-ages were typically around 60 years, and patients with LGI1- and CASPR2-antibodies most frequently had encephalitis or epilepsy. FBDS were exclusive to patients with LGI1-antibodies (p<0.0001) who had more seizures (p=0.01) than patients with CASPR2-antibodies, where peripheral nerve features of neuromyotonia (p=0.0003) and neuropathic pain (p<0.0001) were preferentially associated. As expected, the nine patients with antibodies to intracellular VGKC-epitopes had heterogeneous, often non-immune, clinical syndromes. By contrast, likely non-immune syndromes were noted in only one patient with LGI1-antibodies (stroke) and in four with CASPR2-antibodies (axonal neuropathy, cervical dystonia, hemifacial spasm and psychosis).

Of greater relevance to a HLA study, patients with LGI1- or CASPR2-antibodies often had coexistent autoimmune conditions (28% and 23%, respectively), including Hashimoto's thyroiditis (n=8), psoriasis (n=7) and pernicious anaemia (n=2). Moreover, the LGI1-antibody cohort was distinctive for a 47% rate of ADRs from corticosteroids (p=0.004; 16% with CASPR2-antibodies) and a significantly higher rate of drug-induced rashes in patients with LGI1-antibodies (35% vs 3% in CASPR2, p=0.0004). The reported rashes were secondary to AEDs (n=13: including carbamazepine (n=6), phenytoin (n=4), lamotrigine (n=2) and valproate (n=1), antibiotics (n=6: penicillins (n=5) and metronidazole (n=1)) and immunosuppressants (n=5: azathioprine (n=2), corticosteroids (n=2) and methotrexate (n=1)). Thus, the LGI1- and CASPR2-antibody groups displayed differing clinical autoimmune features suggesting divergent immunogenetic pathways.

Analysis of the HLA allelic profile of patients with LGI1- or CASPR2-antibodies showed strong and distinct HLA allelic profiles as summarised in FIG. 2. Consistent with previous smaller reports almost all LGI1-antibody positive patients carried HLA-DRB1*07:01 (91%, compared to 26% HCs (OR 27.6 (95% CI 12.9-72.2), p=4.1×10-26). Further, 13% (9/68) were homozygous for DRB1*07:01, compared to 2% (115/5553) HCs (OR 7.3 (95% CI 3.3-14.4), p=3×10-4). Alleles recognised to be part of haplotypes involving HLA-DRB1*07:01 were overrepresented, namely HLA-DQA1*02:01, HLA-DQB1*02:02, HLA-DQB1*03:03 and HLA-DPB1*11:01. Additionally, associations were found with two HLA class I alleles, HLA-B*57:01 (OR=3.7 (95% CI 2.0-6.5); p=0.014) and HLA-C*06:02 (OR=3.9 (95% CI 2.4-6.3); p=4.6×10⁻⁵). After conditioning on the commonest allele, HLA-DRB1*07:01, two other DQ alleles reached statistical significance consistent with evidence of an independent association, HLA-DQA1*01:03 (OR=4.4 (95% CI 2.2-8.1); p=4×10⁻³) and HLA-DRB1*01:03 (OR=14.7 (95% CI 3.6-51.5), p=0.04).

In striking contrast, analysis of the CASPR2-antibody group identified a single risk allele; HLA-DRB1*11:01, which was present in 48% of CASPR2-antibody patients compared to 4% of patients with LGI1-antibodies and 9% HCs (OR 9.4 (95% CI 4.6-19.3); p=5.7×10⁻⁶). One CASPR2-antibody patient was homozygous for HLA-DRB1*11:01. Interestingly, the four patients with non-immune conditions and CASPR2-antibodies (FIG. 1) did not carry HLA-DRB1*11:01, giving it a 56% (15/27) frequency in the remainder. No additional alleles were observed after conditioning on HLA-DRB1*11:01.

Intriguingly, of the three patients with coexistent CASPR2 and LGI1-antibodies, only one carried HLA-DRB1*07:01 and none carried HLA-DRB1*11:01. However, all three carried HLA-B*44:02, -C*05:01, -DQA1*03:01 and -DQB1*03:01, a different complement of alleles to the patients with antibodies to either LGI1 or CASPR2.

Haplotype-Specific Distinctions Between Patients with LGI1- and CASPR2-Antibodies

Next, to understand the en bloc allelic inheritance and in vivo relevance of HLA combinations which may present LGI1 and CASPR2 antigens, associations involving HLA haplotypes were explored.

HLA-DQA1*02:01, HLA-DQB1*02:02, HLA-DQB1*03:03 and HLA-DPB1*11:01 showed evidence of linkage disequilibrium with HLA-DRB1*07:01 (r2 values 0.64, 0.49, 0.13, 0.10 and D′ 1, 0.95, 0.8 and 1, respectively (Wang et al., 2005). This was reflected in the most frequent HLA class II haplotypes found in patients with LGI1-antibodies, namely HLA-DRB1*07:01-DQA1*02:01-DQB1*02:02 (OR=5.2 (95% CI 3.2-8.6); p=2.3×10⁻⁹), DRB1*07:01-DQA1*02:01-DQB1*03:03 (OR=3.1 (95% CI 1.7-5.5); p=0.02) and DPA1*02:01-DPB1*11:01 (OR=4.8 (95% CI 2.5-8.5); p=3.8×10⁻⁴). In addition, LGI1-antibody status was associated with a HLA class I haplotype, HLA-C*06:02-B*57:01 (OR=3.6 (95% CI 1.9-6.2); p=8.8×10⁻³). By contrast, only one HLA class II haplotype was associated with CASPR2-antibodies: DRB1*11:01-DQA1*05:01-DQB1*03:01 (OR=7.4 (95% CI 3.5-15.2), p=5.7×10⁻⁵).

Within the LGI1-antibody patients, 5/6 patients with antibiotic-induced rashes carried HLA-B*57:01 known to associate with risk of rash to abacavir and flucloxacillin (Yip et al., 2014), and 4/6 patients with psoriasis harboured the psoriasis risk allele C*06:02 (Arakawa et al., 2015), suggesting the extended haplotypes may explain these specific co-morbidities. Finally, from the nine LGI1- and four CASPR2-antibody patients with a tumour, there were no significant HLA differences compared to non-tumour patients.

It was noted that CASPR2-antibodies are often found in patients with cancer associated immune neurological syndromes.

### Discussion

This study is the first comparative HLA-analysis of LGI1 and CASPR2 autoantibody-mediated diseases, and shows marked and strikingly different HLA-associations for these patients, at both allelic and haplotypic levels. Given the overlapping clinical features in patients with LGI1- and CASPR2-antibodies, and their co-expression in VGKC-complexes, these findings indicate dichotomous predisposing HLA variants govern the generation of LGI1- versus CASPR2-antibodies. Furthermore, they strongly implicate T cells in disease initiation. Strikingly, while HLA-DRB1*07:01 and linked Class II alleles including the haplotype HLA-DRB1*07:01-DQA1*02:01-DQB1*02:02, showed very strong associations with LGI1-antibody patients, this was not observed among CASPR2-antibody patients in whom clear associations with HLA-DRB1*11:01 only were observed. Among LGI1-antibody patients, DRB1*11:01 was observed at around healthy control rates, DRB4 was less frequent than DRB1*07:01, homozygosity for HLA-DRB1*07:01 appeared to confer additional risk and other independent associations involved HLA class I alleles HLA-B*57:01 and HLA-C*06:02. Intriguingly, the patients with both LGI1- and CASPR2-antibodies had yet another complement of HLA-alleles.

Taken together, the range of antigen-restricted peptides derived herein, and the relative HLA-variant frequencies in disease versus control populations, generate hypothesis-driven approaches to expand disease-specific T cells in vitro

In summary, the distinct HLA-associations in patients with LGI1- and CASPR2-autoantibodies, together with differing clinical features relating to autoimmunity, support an immunological dissociation in generation of these clinically-overlapping autoantibody-mediated syndromes.

### Clinical Observations

In clinical studies the responsiveness to immunotherapy of 20 patients with CASPR2-antibodies and HLA-DRB1*11:01 was compared to that of 20 patients with CASPR2-antibodies but without HLA-DRB1*11:01. The patients with CASPR2-antibodies and HLA-DRB1*11:01 consistently showed a positive response to immunotherapy (in 20 out of 20 patients). Whereas patients with CASPR2-antibodies but without HLA-DRB1*11:01 showed a higher chance of having a non-immunotherapy responsive syndrome (7/20, p=0.0083, Fisher's exact test). These results clearly demonstrate the importance of HLA-DRB1*11:01 in the clinical decision-making for patient treatment.

Similarly, in patients with LGI1-antibodies and HLA-DRB1*07:01, there is typically a very good response to immunotherapy. However, from the approximately 10% of LGI1-antibody positive patients who do not carry HLA-DRB1*07:01, there is a higher rate of non-immunotherapy responsive syndromes (lack of immunotherapy-response in 5/10 versus only 2/71 from the group who carry HLA-DRB1*07:01 (p=0.0001, Fisher's exact test).

In addition to the presence of HLA-DRB1*11:01 in patients with CASPR2-antibodies being an indicator of responsiveness to immunotherapy, the clinical data also suggested the presence of a higher overall CASPR-antibody titre (FIG. 6A; FL=to full length CASPR2) and/or the presence of antibodies against the C- and/or N-termini of CASPR2 (CTD and NTD, respectively, FIG. 6B-C) in patients is an indicator of responsiveness to immunotherapy. The absence of HLA-DRB1*11:01 confers a low chance of detecting antibodies against the C or N terminal domains of CASPR2. These results demonstrate that the presence of autoantibodies to the N and/or C terminal ends of CASPR2 are predictive of patients who will respond to immunotherapy, this in addition to or as an alternative to the presence of HLA-DRB1*11:01

Results for the Predictions of HLA-Binding Peptides and their Effectiveness

The robust HLA class II associations identified in this study strongly implicate CD4+ T cells in the pathogenesis of both LGI1- and CASPR2-antibody associated diseases. To locate potentially high-affinity peptides which complex with HLA class II heterodimers, and may interact with patient T cells, in silico modelling was used and focused on all the Class II haplotypes identified (FIG. 5).

Overall, many peptides from both LGI1 and CASPR2 ranked highly for potential binding to several HLA-DR, HLA-DP and HLA-DQ variants (FIG. 5A, C), likely consistent with the varied intrinsic properties of different HLA molecules. Furthermore, for HLA-DRB1*07:01 and -DRB1*11:01, which pair with the invariant DRA chain, and for HLA-DQA1*02:01-DQB1*02:02 heterodimers, peptide ranks showed little difference between LGI1- and CASPR2-derived peptides, suggesting a lack of antigen-selectivity. By contrast, the CASPR2-antibody-associated HLA-DQA1*05:01-DQB1*03:01 heterodimer was predicted to bind some high-ranking peptides from the CASPR2-sequence only, suggesting CASPR2-specificity.

As expected for the shared core sequences between consecutive 15-mers, many highly-ranked peptides were from tightly-clustered locations within the full-length protein (FIG. 5B, D and FIG. 3). Most peptides within these clusters showed potential to bind the HLA variants observed in both the LGI1- and CASPR2-antibody cohorts (black circles; FIG. 5B, D). 9/13 LGI1-derived peptides and 7/13 from the CASPR2 sequence showed binding potential which was more restricted to the variants associated with the corresponding antibody cohort (orange circles, FIG. 5B, D). From LGI1, 4/9 core peptides were predicted to bind with high affinity (<40 nM), typically to HLA-DRB1*07:01, although interestingly the highest affinity peptide was predicted to bind HLA-DPA1*02:01-DPB1*11:01 (FIG. 3). From CASPR2-derived peptides, 7/7 were predicted to bind with high affinity, distributed across the variants within the CASPR2-antibody associated haplotype (FIG. 3).

### Discussion

The data presented shows that select LGI1-derived peptide pools (FIGS. 3 and 4) proliferated patient T cells in vitro (FIG. 4). CDI (normalised to AQP4-peptide proliferation) was elevated using four pools of peptides, suggesting the presence of and stimulation of LGI1-specific T cells by specific peptides derived from the in silico HLA-allele dependent analysis.

The data presented in FIG. 7 demonstrates that 7/11 patients with LGI1-antibodies, 2/6 DC (disease controls) and 2/4 HC (healthy controls) showed a robust CD4⁺ T cell proliferation (CDI≥2) to at least one of the tested LGI1 peptides. Overall, for the majority of the tested peptides, the mean CDI was higher in patients with LGI1 antibodies compared to the other groups, except L3, where the CDI was highest in HC (FIG. 7).

### REFERENCES

Andreatta M, Karosiene E, Rasmussen M, Stryhn A, Buus S, Nielsen M. Accurate pan-specific prediction of peptide-MHC class II binding affinity with improved binding core identification. Immunogenetics 2015; 67: 641-650.
Becker E B E, Zuliani L, Pettingill R, Lang B, Waters P, Dulneva A, et al. Contactin-associated protein-2 antibodies in non-paraneoplastic cerebellar ataxia. J Neuro Neurosurg Psychiatry 2012; 83: 437-440.
Bien C G, Mirzadjanova Z, Baumgartner C, Onugoren M D, Grunwald T, Holtkamp M, et al. Anti-contactin-associated protein-2 encephalitis: relevance of antibody titres, presentation and outcome. Eur J Neurol 2017; 24: 175-186.
Binks S N M, Klein C J, Waters P, Pittock S J, Irani S R. LGI1, CASPR2 and related antibodies: a molecular evolution of the phenotypes. J Neuro Neurosurg Psychiatry 2017: jnnp-2017-315720.
Bralten L B C, Gravendeel A M, Kloosterhof N K, Sacchetti A, Vrijenhoek T, Veltman J A, et al. The CASPR2 cell adhesion molecule functions as a tumor suppressor gene in glioma. 2010; 29: 6138-6148.
Bunce M, O'Neill C M, Barnardo M C, Krausa P, Browning M J, Morris P J, et al. Phototyping: comprehensive DNA typing for HLA-A, B, C, DRB1, DRB3, DRB4, DRB5 & DQB1 by PCR with 144 primer mixes utilizing sequence-specific primers (PCR-SSP). Tissue Antigens 1995; 46: 355-367.
Gadoth A, Pittock S J, Dubey D, McKeon A, Britton J W, Schmeling J E, et al. Expanded phenotypes and outcomes among 256 LGI1/CASPR2-IgG positive patients. Ann Neurol. 2017; 82: 79-92.
Irani S R, Alexander S, Waters P, Kleopa K A, Pettingill P, Zuliani L, et al. Antibodies to Kv1 potassium channel-complex proteins leucine-rich, glioma inactivated 1 protein and contactin-associated protein-2 in limbic encephalitis, Morvan's syndrome and acquired neuromyotonia. Brain 2010; 133: 2734-2748.
Irani S R, Michell A W, Lang B, Pettingill P, Waters P, Johnson M R, et al. Faciobrachial dystonic seizures precede Lgi1 antibody limbic encephalitis. Ann Neurol. 2011; 69: 892-900.
Irani S R, Stagg C J, Schott J M, Rosenthal C R, Schneider S A, Pettingill P, et al. Faciobrachial dystonic seizures: the influence of immunotherapy on seizure control and prevention of cognitive impairment in a broadening phenotype. Brain 2013; 136: 3151-3162.
Lai M, Huijbers M G M, Lancaster E, Graus F, Bataller L, Balice-Gordon R, et al. Investigation of LGI1 as the antigen in limbic encephalitis previously attributed to potassium channels: a case series. Lancet Neurol 2010; 9: 776-785.
Lang B, Makuch M, Moloney T, Dettmann I, Mindorf S, Probst C, et al. Intracellular and non-neuronal targets of voltage-gated potassium channel complex antibodies. J Neuro Neurosurg Psychiatry 2017; 88: 353-361.
Podewils von F, Suesse M, Geithner J, Gaida B, Wang Z I, Lange J, et al. Prevalence and outcome of late-onset seizures due to autoimmune etiology: A prospective observational population-based cohort study. Epilepsia 2017; 58: 1542-1550.
Thompson J, Bi M, Murchison A G, Makuch M, Bien C G, Chu K, et al. The importance of early immunotherapy in patients with faciobrachial dystonic seizures. Brain 2018; 141: 348-356.

1. A method of identifying patients who are positive for CASPR2 autoantibodies which are predicted to respond to immunotherapy, the method comprising:
i) obtaining a sample from a subject having autoantibodies against CASPR2;
ii) screening for the presence of the allele HLA DRB1*11:01.

2. The method of claim 1 further comprising the step of concluding that if the HLA DRB1*11:01 allele is present then immunotherapy should be administered.

3. A method of stratifying subjects into those which are predicted to respond to immunotherapy, the method comprising:
i) obtaining a sample from a subject having autoantibodies against CASPR2;
ii) screening for the presence of the allele HLA DRB1*11:01;
iii) predicting if HLA DRB1*11:01 allele is present that the subject will respond to immunotherapy.

4. A method of diagnosing in a mammal an autoimmune neurological disorder, the method comprising:
i) obtaining a sample provided by a subject;
ii) detecting the presence or absence of CASPR2 autoantibodies in the sample;
iii) detecting the presence or absence of the HLA DRB1*11:01 allele in the subject; and optionally
iv) concluding that if CASPR2 autoantibodies are present and the HLA DRB1*11:01 allele is present that the subject has an autoimmune neurological disorder.

5. The method of claim 4, wherein the autoimmune neurological disorder may be selected from the group consisting of limbic encephalitis, Morvan's syndrome, neuromyotonia, and other increasingly recognised associations of CASPR2-antibodies, including movement disorders such as ataxia and myoclonic syndromes, pain syndromes and forms of epilepsy.

6. The method of any preceding claim further comprising screening for one or more of the following:
the titre of CASPR2 autoantibodies; and
the IgG subclass of CASPR2 autoantibodies;
the presence of CASPR2 autoantibodies in solution, which may be determined by a fluorescent immunoprecipitation assay (FIPA).

7. The method of claim 6, wherein if the titre of CASPR2 autoantibodies is high, it is concluded that the autoantibodies are disease causing and the subject should be treated accordingly, typically with immunotherapy.

8. The method of claim 6, wherein if the titre of CASPR2 autoantibodies is low, it is indicated that the autoantibodies are not disease causing

9. The method of any preceding claim where if the CASPR2 autoantibody is IgG4, this is indicative that the autoantibodies are causative of disease.

10. The method of any preceding claim, where the sample comprises one or more of plasma, serum, whole blood, urine, sweat, tears, saliva, lymph, faeces, cerebrospinal fluid, and nipple aspirate.

11. A method for predicting whether or not an individual will respond to immunotherapy, wherein the method comprises determining whether a subject has CASPR2 autoantibodies and whether a subject has HLA DRB1*11:01 allele, and if both are present it is indicated that the subject will respond to immunotherapy.

12. A method of treating an autoimmune neurological disorder in an individual, the method comprising:
i) diagnosing an autoimmune neurological disorder according to the method above; and
ii) administering to the individual an agent useful in the treatment of the autoimmune neurological disorder.

13. A system comprising:
a) a measuring module for determining the presence of CASPR2 autoantibodies in a biological sample from a subject;
b) a measuring module for determining the presence of the HLA DRB1*11:01 allele in a biological sample from a subject.
c) a storage module configured to store data output from the measuring module or modules, and optionally reference data;
d) a computation module configured to compute the value of the data output from the measuring module or modules, and optionally the reference data; and
e) an output module configured to display a diagnosis for the subject based on the results obtained by the computation module.

14. An assay kit for diagnosing, in a mammal, an autoimmune neurological disorder selected from the group comprising limbic encephalitis, Morvan's syndrome, neuromyotonia, and other conditions according to the method of the invention, wherein the kit comprises at least one epitope of CASPR2 and primers for use in detecting the HLA DRB1*11:01 allele, and instructions to use the kit.

15. One or more novel isolated peptides, wherein the peptide comprises the sequence of any of Seq ID no: 1 to Seq ID no: 57 or a sequence having at least 80%, 85%, 90%, 95% or more sequence identity with one of Seq ID no 1 to 57, preferably with one of Seq ID no 40 to 57.

16. The peptides of claim 15 comprising one or more conservative amino acid substitutions.

17. A pharmaceutical composition comprising a peptide of claim 15 or 16 and a pharmaceutically acceptable carrier, diluent or excipient.

18. A method of treating a subject with an autoimmune neurological condition comprising administering to the subject an effective amount of a peptide according to claim 15 or 16 or a pharmaceutical composition according to claim 17.

19. A peptide according to claim 15 or 16 or a pharmaceutical composition according to claim 17 for use in of treating an autoimmune neurological condition.
